(19) United States
(12) Patent Application Publication
(10) Pub. No.: US 2019/0060410 A1
(43) Pub. Date: Feb. 28, 2019

(54) Pharmaceutical Formulation Comprising GLP-1 Analogue and Preparation Method Thereof

(71) Applicants: Xiaoni Liu, Hangzhou, Zhejiang (CN); Hanxing Li, Hangzhou, Zhejiang (CN); Chaoliang CHEN, Hangzhou, Zhejiang (CN); Guochang Ma, Hangzhou, Zhejiang (CN); Feihu Xu, Hangzhou, Zhejiang (CN); Tongying Wang, Hangzhou, Zhejiang (CN); Handong Sun, Hangzhou, Zhejiang (CN); Hangzhou Jiuyuan Gene Engineering Co., Ltd., Hangzhou, Zhejiang (CN)

(72) Inventors: Xiaoni Liu, Zhejiang (CN); Hanxing Li, Zhejiang (CN); Chaoliang CHEN, Zhejiang (CN); Guochang Ma, Zhejiang (CN); Feihu Xu, Zhejiang (CN); Tongying Wang, Zhejiang (CN); Handong Sun, Zhejiang (CN)

(21) Appl. No.: 15/571,278

(22) PCT Filed: May 12, 2016
(86) PCT No.: PCT/CN2016/081843

§ 371 Date: Nov. 2, 2017

(30) Foreign Application Priority Data
May 13, 2015 (CN) 201510243040.3

(51) Int. Cl. A61K 38/26; A61K 47/10; A61K 47/26; A61K 47/02; A61K 9/00

(52) U.S. Cl. CPC A61K 38/26; A61K 47/10; A61K 9/0019; A61K 47/02; A61K 47/26

(57) ABSTRACT

The present invention discloses a pharmaceutical composition comprising GLP-1 analogs. In one embodiment, the composition further comprises buffers, stabilizers, isotonic agents, preservatives, or a mixture thereof. The present invention has the advantage of providing a highly stabilized pharmaceutical formulation of a GLP-1 analog suitable for long-term shelf-life and distribution in the commercial pharmaceutical supply chain. The disclosed formulations effectively protect the active ingredient GLP-1 analogs from degradation, oxidation, precipitation, crystallization, and other factors leading to loss of clinical efficacy.

### FIELD OF THE INVENTION

The present invention relates to a pharmaceutical formulation comprising a peptide drug. More particularly, the present invention relates to a pharmaceutical preparation comprising a GLP-1 analog and a process for the preparation thereof.

### BACKGROUND OF THE INVENTION

In the 1960s, McIntyre and Elrick found that the effect of oral glucose on insulin secretion was significantly higher than that of intravenous injection, and this additional effect was called “incretin effect”. With the development of cell and molecular biology, studies have confirmed that incretin is an important human intestinal hormone. Following eating, the hoitnone promotes insulin secretion and exerts a glucose-dependent hypoglycemic effect.

Incretin is mainly composed of Glucagon-like peptide-1 (GLP-1) and glucose-dependent insulinotropic polypeptide (GIP). Both peptides are rapidly expressed following nutrient ingestion. The GLP-1 component plays the more important role in the development and progression of type 2 diabetes mellitus. Studies have shown that GLP-1 reduces blood glucose by stimulating insulin secretion, inhibiting glucagon secretion and modulating gastric emptying. Additionally, GLP-1 has a unique role in slowing beta cell apoptosis and promoting beta cell regeneration.

In 1983, McIntyre et al. identified GLP-1 in the analysis of the gene sequence of glucagon precursor (proglucagon, PG). The GLP-1 gene is expressed in pancreatic α-cells and intestinal L cells. The complete GLP-1 polypeptide is 37 amino acids with the following peptide sequence:

HDEFERHAEGTFTSDVSSYLEGQAAKEFIAWLVKGRG;

In vivo, the bioactive forms of GLP-1 are the GLP-1-(7-37) peptide and the GLP-1-(7-36)-amide peptide. About 80% of GLP-1 activity is due to the GLP-1-(7-36)-amide peptide. As is customary in the art, the amino terminus of GLP-1 (7-37)-OH is designated residue number 7 and the carboxy terminus is designated residue 37. For a more detailed description of GLP-1 analogs and derivatives, see Hoffmann, J A [WO1999029336, published Jun. 17, 1999] and Knudsen, L B et al. [J. Med. Chem. 43: 1664-1669 (2000)]. After GLP-1 (1-37) was formed in vivo, the N-terminal 6 amino acids were removed and the C-terminal amide was &limed by two-step Enzyme digestion to finally generate the highly active GLP-1 (7-36) amide (also known as the GLP-1 fragment).

GLP-1 is the most important intestinal peptide hormone that promotes insulin secretion. GLP-1 acts by binding to the GLP-1 receptor (GLP-1R, a G-coupled protein belonging to the β-receptor family), triggering intracellular cyclic adenosine monophosphate (cAMP) production and stimulating the mitogen-activated protein kinase (MAPK) pathway. Mature islet beta cells respond to GLP-1 binding to GLP-1R by activation of adenylyl cyclase through stimulatory G-protein Gs, which increases cAMP production. The increased cAMP production cascades to further signaling so that GLP-1 and glucose synergistically stimulate insulin gene transcription, insulin protein synthesis and insulin secretion. Additionally, the signaling reduces the concentration of glucagon, inhibits glucagon secretion, enhances cell sensitivity to insulin, stimulates insulin-dependent glycogen synthesis, and eventually reduces postprandial blood glucose levels. By activating phosphatidylinositol 3-kinase (PI3K) and MAPK pathway, GLP-1 regulates pre-apoptotic proteins and induces the expression of the anti-apoptotic proteins Bcl-2 and Bcl-xL. These two proteins act to reduce beta-cell apoptosis, enhance beta-cell regeneration, and promote islet beta-cell differentiation and proliferation. In addition, GLP-1 slows gastric emptying, and suppresses appetite by acting on the hypothalamus.

At present in the industry, GLP-1 and its analogs are expressed as recombinant proteins in a bacterial or fungal cell systems, then purified and further processed. Liraglutide is one GLP-1 analog has been approved for human therapeutic use and is marketed under the tradename Victoza. Liraglutide bears a C16-fatty acid modification linked to Lysine 26 through a glutamic acid spacer; chemically, liraglutide is Arg³⁴, Lys²⁶ (N^{ε}-(γ-Glu (N^{α}-hexadecanoyl))) GLP-1 (7-37) as indicated in the figure below. The fatty acid modification extends liraglutide's plasma half-life to 13 hours following subcutaneous injection. Liraglutide's GLP-1 sequence is similar to human GLP-1 (7-37) sequence, bearing a 97% homology. In addition to the modification at Lysine 26, Lysine 34 is replaced with an arginine residue (Goke et al., J. Biol. Chem., 268:

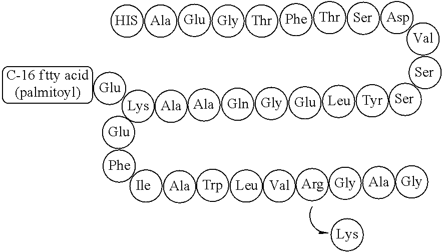

Upon administration, liraglutide has the following pharmacological effects due to high homology with GLP-1: (1) enhanced insulin secretion by blood glucose concentration-dependent manner; (2) inhibited postprandial glucagon secretion; (3) suppressed appetite, and slowed gastric emptying. In addition, liraglutide can promote beta cell proliferation and differentiation, regulate beta cell apoptosis gene-related expression and inhibit apoptosis. Together, the combined effects of reduced apoptosis, increased proliferation and differentiation, increases the number of pancreatic islet beta cells. Studies have further shown that liraglutide can also restore the sensitivity of human islet beta cells to circulating blood glucose.

Typically, oral hypoglycemic drugs are the first-line treatment for type 2 diabetes; however, due to the poor patient tolerance or other side effects, in particular hypoglycemia, not all patients with type 2 diabetes may be treated with this first-line class. For these and similar patients with uncontrolled type 2 diabetes, liraglutide has been shown to be a promising alternative therapeutic. The LEAD (Liraglutide Effect and Action in Diabetes) series studies evaluated liraglutide alone and in combination with other oral antidiabetic drugs. Combined, the LEAD studies encompassed more than 5,000 diabetic patients in more than 40 countries. The data showed that liraglutide protects islet beta cell function and improve the quality and the amount of insulin secreted by the beta cells. This demonstrated biologic effect shows the potential of liraglutide to alter the clinical trajectory of type 2 diabetes.

The LEAD studies included six phase III clinical trials extensively evaluating the efficacy of liraglutide monotherapy and combination therapy with other oral hypoglycemic drugs. These studies demonstrate that liraglutide can not only effectively protect the function of islet beta cells and delay the clinical progress of type 2 diabetes, but also rapidly, efficiently and permanently reduce glycosylated hemoglobin (HbA1c) and better control serum glucose levels preventing hypoglycemia. Liraglutide showed better glycemic control than the comparison arms in these studies. Additionally, the studies demonstrated a statistically significant weight loss sustained for up to two years and reduction of systolic blood pressure.

The LEAD series clinical trials revealed that liraglutide has a protective cardiovascular effect. Liraglutide can reduce systolic blood pressure by 2.7-6.7 mmHg; this cardiovascular effect occurs before the weight loss, so the systolic blood pressure effect is not fully explained by the patient's weight loss. Additionally, patients receiving liraglutide in the studies reported slightly increased resting pulse rates. This data further suggests a cardiovascular effect; however, the clinical significance of these pulse rate changes remains unclear.

Courreges et al. reported that liraglutide treatment modulated patient serum lipid levels. They reported decreases in total cholesterol, low density lipoprotein, free fatty acid, triglyceride, plasminogen activator inhibitor-1 and B-type natriuretic peptide concentrations. Courreges also reported dose-dependent reductions in high-sensitivity C-reactive protein, although the magnitude of these changes may not have been significant.

Liraglutide's glucose-lowering effect is itself glucose-dependent, promoting insulin secretion and inhibiting glucagon action only when the blood glucose concentration is high. Consequently, liraglutide treatment alone produces almost no hypoglycemia. Clinical trials have shown that hypoglycemia accompanying liraglutide monotherapy is significantly less frequent than hypoglycemia accompanying glimepiride treatment, and comparable to frequency with metformin treatment. Liraglutide combination therapy prevents the majority of hypoglycemia events; the event frequency is similar to or less than other hypoglycemic drugs. Nauck et al. reported that hypoglycemia in patients treated with liraglutide combined with metformin was comparable to the hypoglycemia rate in placebo plus metformin patients (less than 3%). The incidence of hypoglycemia was 17% in Patients treated with glimepiride and metforminhad a 17% hypoglycemia incidence; the difference between the liraglutide/metformin rate and the glimepiride/metformin was statistically significant (P<0.001). The liraglutide/metformin combination showed better glycemic control in these studies.

The most common adverse reactions in liraglutide treatment are gastrointestinal reactions, mainly manifestations of nausea, vomiting, and diarrhea. These adverse reactions are usually observed during the first week of treatment and are dose-dependent. In the LEAD-2 and LEAD-3 clinical trials, fewer than 10% of liraglutide treated patients reported nausea. When the liraglutide dose is slowly titrated up over the first three weeks of treatment, patients showed a much lower incidence of gastrointestinal side effects. Only a few patients discontinued treatment because of gastrointestinal symptoms when the slow titration initial regimen was used.

Liraglutide, alone or in combination with other oral hypoglycemic drugs, can quickly and efficiently reduce HbA1c levels and provide good glycemic control. Because liraglutide's hypoglycemic effect depends on serum glucose concentration and is coupled to insulin release, the probability of breakthrough hypoglycemia is very low. Liraglutide's potential to slow the clinical progression of diabetes is noteworthy: A large number of clinical trials have shown that liraglutide improves beta cell function, reduces beta cell apoptosis, and increases beta cell differentiation. The protective effect of liraglutide on the cardiovascular system allows it to reduce the incidence of cardiovascular complications associated with diabetes. In addition, liraglutide has a significant effect on weight loss making it suitable for patients with severe a hypoglycemia and who need to lose weight. Together, liraglutide's unique pharmacological effects have broad applicability to the treatment of diabetes.

Chinese Patents 97198413.1 and 99808706.8 report the liraglutide production process. The intrinsic properties of GLP-1 protein make it susceptible to a wide variety of hydrolytic enzymes that can rapidly degrade the protein (M. Egel-Mitani, et al., Yield improvement of heterologous peptides expressed in ypsl-disrupted Saccharomyces cerevisiae lines, Enzyme and Microbial Technology 26:671-677 (2000)). The fatty acid modification to residue 26 increases liraglutide stability compared to the wild-type, unmodified form of the peptide. Because liraglutide is a peptide therapeutic, environmental factors will affect its stability in long-term storage and in the commercial supply chain.

Liraglutide is highly sensitive to temperature, oxygen and ultraviolet light. Significant temperature changes, high dissolved oxygen levels or exposure to ultraviolet light may trigger a variety of unfavorable physical or chemical changes. These unfavorable changes include adsorption to the container walls, polymerization, precipitation and oxidation. This sensitivity to environmental and storage conditions requires that the formulation be carefully selected to ensure 90% purity over the formulation's shelf life. Proper formulation is essential to maintain liraglutide stability and ensure delivery of a suitable effective clinical dosage of the active peptide.

Visible foreign matter in the formulation solution, that is, a precipitate, may form if the formulation is physically disturbed. Vibration may stimulate aggregation of the peptide at the liquid-gas interface. It is believed that protein molecules are adsorbed on the gas-liquid interface with their hydrophobic groups extended out of the aqueous phase and into the overtopping gas phase and keeping their hydrophilic groups immersed in the aqueous phase. Once so arranged on the liquid surface, the protein molecules nucleate and then form particles and eventually form visible precipitates. Mechanical disturbance of the solution during shipping may cause conformational changes in the proteins adsorbed at the gas-liquid and solid-liquid interfaces. This mechanical disturbance may cause protein entanglement or aggregation, forming particles and eventually visible precipitate. In addition, visible particulates may form as a result of the freeze-drying process due to various factors like the pre-freeze rate, heating rate, or other freeze-drying process parameters.

Chinese patent application CN200480034152.8 refers to an improved formulation of liraglutide. In the past experiments it has been found that the liraglutide is easily crystallized, even from the pharmaceutical formulation. The disclosed mannitol formulation readily produced crystals. These crystals clogged production devices and needles affecting both production and clinical use. A variety of agents, including mannitol, glycerol, sucrose, PEG400, arginine, dimethylsulfone, sorbitol, inositol, glucose, glycine, maltose and lactose were excluded as less ideal; however, propylene glycol was ultimately selected as an isotonic regulator in the liraglutide formulation. Propylene glycol had no negative effect on the physical stability or on the chemical stability of liraglutide (but no data was provided). These formulations did not readily form visible precipitates or sediments.

Because liraglutide is intended to treat diabetes, a chronic disease, a patient will be using the liraglutide formulation every day over a very long time period. Although propylene glycol is relatively safe and well tolerated when used as an adjuvant and infrequently, there are risks to long-term systemic use:

(1) skin irritation: may induce the subjective sense of burning, tingling and itching during use;

(2) Defatting: long-term use of high concentrations of propylene glycol may have an impact on the skin sebum structure;

(3) irritant-based dermatitis: propylene glycol will irritate the skin and mucous membranes, at higher the concentrations it may cause skin redness, rash, peeling itching and rough situation. Although for the majority of patients, there is little or no reaction; however, with prolonged use, there may be a cumulative effect, increasing the probability of dermatitis;

(4) allergic dermatitis: about 1 to 5% of people exposed to propylene glycol will produce local skin allergic eczema reaction once sensitized; further exposure may result in local allergic dermatitis; and

(5) systemic contact dermatitis: A small number of people who are skin sensitized to propylene glycol, may have systemic allergic reactions when given drugs containing propylene glycol.

Despite its general safety and ability to be tolerated in low infrequent exposures, there is still the potential for local irritation, sensitization and systemic reactions. Because of this liability, alternative formulations with less or no propylene glycol are preferable.

Therefore, it has become extremely important to develop a pharmaceutical preparation where the propylene glycol is replaced with a different pharmaceutical excipient. The present invention is an alternative formulation of lirglutide with such properties.

### DESCRIPTION OF THE INVENTION

This invention embodies a stable pharmaceutical composition a comprising a GLP-1 analog. The present invention relates to pharmaceutical formulations comprising GLP-1 analog in a concentration from 0.1 mg/ml to 25 mg/ml, a buffer with pH from 7.5 to 9.0, a stabilizer in a concentration from 0.001% to 0.5% (m/v), xylitol in a concentration from 0.5% to 10%(m/v), and a preservative in a concentration from 0.1 mg/ml to 10 mg/ml.

In the present invention, the teen “GLP-1 analog” is understood to refer to GLP-1 and any mutant thereof, GLP-1(7-36)-amide and any mutant thereof, GLP-1 (7-37) and any mutant thereof, and chemically modified “GLP-1 analog” derivatives in which organic substituents have been added to one or more amino acid residues of GLP-1 analog peptide.

The term “mutant” is used to designate the parent peptide GLP-1, GLP-1 (7-36)-amide and GLP-1 (7-37) wherein one or more amino acid residues of the parent, wild-type peptide have been substituted with another amino acid residue and/or wherein one or more amino acid residues of the parent peptide have been deleted and/or wherein one or more amino acid residues have been added to the parent peptide. Such addition can take place either at the N-terminal end or at the C-terminal end of the parent peptide or both.

Preferably, the mutant is a peptide wherein 6 or fewer amino acids have been substituted and/or added and/or deleted from the parent peptide. More preferably, a peptide wherein 3 or fewer amino acids have been substituted and/or added and/or deleted from the parent peptide. Most preferably, a peptide wherein one amino acid has been substituted and/or added and/or deleted from the parent peptide.

In one embodiment, the mutant means the parent peptide GLP-1, GLP-1 (7-36)-amide or GLP-1 (7-37) wherein one or more amino acid residues of the parent peptide have been substituted by another amino acid residue and/or wherein one or more amino acid residues of the parent peptide have been deleted and/or wherein one or more amino acid residues have been added to the parent peptide.

In one embodiment of the invention, the GLP-1 derivative preferably has three lipophilic substituents, more preferably two lipophilic substituents, and most preferably one lipophilic substituent attached to the parent peptide (e.g. GLP-1(7-36)-amide, GLP-1(7-37), a GLP-1(7-36)-amide analog or a GLP-1(7-37) analog), where each lipophilic substituent(s) preferably has 4-40 carbon atoms, more preferably 8-30 carbon atoms, even more preferably 8-25 carbon atoms, even more preferably 12-25 carbon atoms, and most preferably 14-18 carbon atoms.

In one embodiment, the GLP-1 derivative is chemically modified by introducing an organic substituent e.g. GLP-1 analogs suitable for the present invention are described in the prior art which includes those referred to WO93/19175 (Novo Nordisk), WO99/43705 (Novo Nordisk), WO99/43706 (Novo Nordisk), WO99/43707 (Novo Nordisk), WO98/08871 (analogs with lipophilic substituents) and in WO02/46227 (analogs fused to serum albumin or to Fc portion of an Ig) (Novo Nordisk A/S), WO99/43708 (Novo Nordisk A/S), WO99/43341 (Novo Nordisk A/S), WO87/06941 (The General Hospital Corporation), WO90/11296(The General Hospital Corporation), WO91/11457(Buckley et al.), WO98/43658 (Eli Lilly & Co.), EP0708179-A2 (Eli Lilly & Co.), EP0699686-A2 (Eli Lilly & Co.), WO01/98331(Eli Lilly & Co.), and CN200480034152.8; those analogs disclosed therein are expressly incorporated by reference in their entirety.

In one embodiment of the present invention, the GLP-1 analog is preferably Arg³⁴,Lys²⁶(N^{ε}-(γ-Glu(N^{α}-hexadecanoyl)))-GLP-1(7-37), and is referred to as Liraglutide.

In yet another embodiment of the present invention, the GLP-1 analog is selected from the group consisting of Gly⁸-GLP-1(7-36)-amide, Gly⁸-GLP-1(7-37), Val⁸-GLP-1(7-36)-amide, Val⁸-GLP-1(7-37), Val⁸Asp²²-GLP-(7-36)-amide, Val⁸Asp²²-GLP-1(7-37), Val⁸Glu²²-GLP-1 (7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1 (7-37), Val⁸His²²-GLP-1(7-36)-amide, Val⁸His²²-GLP-1(7-37), analogs thereof and derivatives of any of these.

In yet another embodiment of the present invention, the GLP-1 analog is selected from the group consisting of Arg²⁶-GLP-1(7-37); Arg³⁴-GLP-1(7-37); Lys³⁶-GLP-1(7-37); Arg^{26,34}Lys³⁶-GLP-1(7-37); Arg^{26,34}-GLP-1(7-37); Arg^{26,34}Lys⁴⁰-GLP-1(7-37); Arg²⁶Lys³⁶-GLP-1(7-37); Arg³⁴Lys³⁶-GLP-1(7-37); Val⁸Arg²²-GLP-1(7-37); Met⁸Arg²²-GLP-1(7-37); Gly⁸His²²-GLP-1(7-37); Val⁸His²²-GLP-1(7-37); Met⁸His²²-GLP-1 (7-37); His³⁷-GLP-1(7-37); Gly⁸-GLP-1(7-37); Val⁸-GLP-1(7-37); Met⁸-GLP-1(7-37); Gly⁸Asp²²-GLP-1(7-37); Val⁸Asp²²-GLP-1(7-37); Met⁸Asp²²-GLP-1(7-37); Gly⁸Glu²²-GLP-1(7-37); Val⁸Glu²²-GLP-1(7-37); Met⁸Glu²²-GLP-1(7-37); Gly⁸Lys²²-GLP-1(7-37); Val⁸Lys²²-GLP-1(7-37); Met⁸Lys²²-GLP-1(7-37); Gly⁸Arg²²-GLP-1(7-37); Val⁸Lys²²His³⁷-GLP-1 (7-37); Gly⁸Glu²²His³⁷-GLP-1(7-37); Val⁸Glu²²His³⁷-GLP-1(7-37); Met⁸Glu²²His³⁷-GLP-1(7-37); Gly⁸Lys²²His³⁷-GLP-1(7-37); Met⁸Lys²²His³⁷-GLP-1(7-37); Gly⁸Arg²²His³⁷-GLP-1(7-37); Val⁸Arg²²His³⁷-GLP-1 (7-37); Met⁸Arg²²His³⁷-GLP-1(7-37); Gly⁸His²²His³⁷-GLP-1(7-37); Val⁸His²²His³⁷-GLP-1(7-37); Met⁸His³³His³⁷-GLP-1(7-37); Gly⁸His³⁷-GLP-1(7-37); Val⁸His³⁷-GLP-1(7-37); Met⁸His³⁷-GLP-1(7-37); Gly⁸Asp²²His³⁷-GLP-1(7-37); Val⁸Asp²²His³⁷-GLP-1 (7-37); Met⁸Asp²²His³⁷-GLP-1(7-37); Arg²⁶-GLP-1(7-36)-amide; Arg³⁴-GLP-1(7-36)-amide; Lys³⁶-GLP-1(7-36)-amide; Arg^{26,34}Lys³⁶-GLP-1(7-36)-amide; Arg^{26, 34}-GLP-1(7-36)-amide, Arg^{26,34}Lys⁴⁰-GLP-1(7-36)-amide; Arg²⁶Lys³⁶-GLP-1(7-36)-amide; Arg³⁴Lys³⁶-GLP-1(7-36)-amide; Gly⁸-GLP-1(7-36)-amide; Val⁸-GLP-1(7-36)-amide; Met⁸-GLP-1(7-36)-amide; Gly⁸Asp²²-GLP-1(7-36)-amide; Gly⁸Glu²²His³⁷-GLP-1(7-36)-amide; Val⁸Asp²²-GLP-1(7-36)-amide; Met⁸Asp²²-GLP-1(7-36)-amide; Gly⁸Glu²²-GLP-1(7-36)-amide; Val⁸Glu²²-GLP-1(7-36)-amide; Met⁸Glu²²-GLP-1(7-36)-amide; Gly⁸Lys²²-GLP-1 (7-36)-amide; Val⁸Lys²²-GLP-1(7-36)-amide; Met⁸Lys²²-GLP-1(7-36)-amide; Gly⁸His²²His³⁷-GLP-1(7-36)-amide; Gly⁸Arg²²-GLP-1(7-36)-amide; Val⁸Arg²²-GLP-1(7-36)-amide; Met⁸Arg²²-GLP-1(7-36)-amide; Gly⁸His²²-GLP-1(7-36)-amide; Val⁸His²²-GLP-1(7-36)-amide; Met⁸His²²-GLP-1(7-36)-amide; His³⁷-GLP-1(7-36)-amide; Val⁸Arg²²His³⁷-GLP-1(7-36)-amide; Met⁸Arg²²His³⁷-GLP-1(7-36)-amide; Gly⁸His³⁷-GLP-1(7-36)-amide; Val⁸His³⁷-GLP-1(7-36)-amide; Met⁸His³⁷-GLP-1(7-36)-amide; Gly⁸Asp²²His³⁷-GLP-1(7-36)-amide; Val⁸Asp²²His³⁷-GLP-1(7-36)-amide; Met⁸Asp²²His³⁷-GLP-1(7-36)-amide; Val⁸Glu²²His³⁷-GLP-1(7-36)-amide; Met⁸Glu²²His³⁷-GLP-1 (7-36)-amide; Gly⁸Lys²²His³⁷-GLP-1(7-36)-amide; Val⁸Lys²²His³⁷-GLP-1(7-36)-amide; Met⁸Lys²²His³⁷-GLP-1(7-36)-amide ; Gly⁸Arg²²His³⁷-GLP-1(7-36)-amide; Val⁸His²²His³⁷-GLP-1 (7-36)-amide; Met⁸His²²His³⁷-GLP-1 (7-36)-amide; and derivatives thereof.

In yet another embodiment of the present invention, the GLP-1 analog is selected from the group consisting of Val⁸Trp¹⁹Glu²²-GLP-1(7-37), Val⁸Glu²²Val²⁵-GLP-1(7-37), Val⁸Tyr¹⁶Glu²²-GLP-1(7-37), Val⁸Trp¹⁶Glu²²-GLP-1(7-37), Val⁸Leu¹⁶Glu²²-GLP-1 (7-37), Val⁸Tyr¹⁸Glu²²-GLP-1(7-37), Val⁸Glu²²His³⁷GLP-1(7-37), Val⁸Glu²²Ile³³-GLP-1(7-37), Val⁸Trp¹⁶Glu²²Val²⁵Ile³³-GLP-1(7-37), Val⁸Trp¹⁶Glu²²Ile³³-GLP-1(7-37), Val⁸Glu²²Val²⁵Ile³³-GLP-1(7-37), Val⁸Trp¹⁶Glu²²Val²⁵-GLP-1(7-37), analogs thereof, and derivatives thereof.

In the pharmaceutical composition disclosed in the present invention, the concentration of the GLP-1 analog is preferably about 1 mg/ml to 15 mg/ml, more preferably 3 mg/ml to 10 mg/ml, and most preferably 6 mg/ml.

In the pharmaceutical composition disclosed in the present invention, the buffer suitable for use in the present invention is any buffer capable of maintaining the pH of the formulation in the aqueous solution at a pH from 7.5 to 9.0, which can be selected from the group consisting of phosphate buffer, a disodium hydrogen phosphate-citrate buffer, TRIS buffer, glycyl-glycine buffer, N-bis (hydroxyethyl) glycine buffer, sodium dihydrogen phosphate buffer, disodium hydrogen phosphate buffer, sodium acetate buffer, sodium carbonate buffer, sodium phosphate buffer, lysine buffer, arginine buffer and a mixture of any of these thereof.

The pH of the buffer is preferably in the range of 7.5 to 8.5, more preferably 8.0 to 8.5; the concentration of the buffer is 5 to 100 mmol/L, preferably 10 to 30 mmol/L. Preferably the buffer is the disodium hydrogen phosphate buffer which concentration is in the range of 5 to 100 mmol/L and the pH is in the range of 7.5 to 8.5; more preferably the concentration is in the range of 10 to 30 mmol/L and the pH is in the range of 8.0 to 8.5.

In the pharmaceutical composition disclosed in the present invention, xylitol is used as an isotonicity regulator, and the concentration of such regulator is in the range of 0.5% to 10% (m/v), more preferably in the range of 1% to 5% (m/v).

In the pha maceutical composition disclosed in the present invention, stabilizers for improving the stability of GLP-1 analogs include, but are not limited to: amino acids and amino acid derivatives: glycine, alanine, serine, aspartic acid, glutamic acid, threonine, tryptophan, lysine, hydroxy lysine, histidine, arginine, cystine, cysteine, methionine, phenylalanine, leucine, isoleucine amino acids and their derivatives; nonionic surfactants: sorbitan fatty acid esters, glycerol fatty acid esters (e.g., sorbitan monoate, sorbitan monolaurate and sorbitan palm acid monoester), polyglycerol fatty acid esters (e.g., glyceryl octanoic acid monoester, glyceryl myristate mono-tallow cream and glycerol hard fatty acid monoester), polyoxyethylene sorbitan fatty acid esters, polyoxyethylene sorbitol fatty acid esters, polyoxyethylene glycerol fatty acid esters, polyoxyethylene glycol fatty acid esters, polyoxyethylene alkyl ethers, polyoxyethylene polyoxypropylene alkyl ethers, polyoxyethylene phenyl ethers, polyoxyethylated hard castor oil, polyoxyethylated beeswax derivatives, polyoxyethylenated lanolin derivatives or a polyoxyethylene fatty acid amide, wherein the cationic surfactant is an alkyl sulfate (e.g., a C10-C18 alkyl alkyl sulfate); polyethylene glycol, polyvinyl alcohol, hydroxypropyl-dextrins, carboxymethylcellulose, polyvinylpyrrolidone, polysorbate 20, polysorbate 80, or any poloxamer seriesmolecules (e.g. poloxamer 124, poloxamer 181, poloxamer 182, poloxamer 188, poloxamer 237, poloxamer 331, poloxamer 338 or poloxamer 407).

The stabilizers are preferably polysorbate 20, polysorbate 80, poloxamer 124, poloxamer 181, poloxamer 182, poloxamer 188, poloxamer 237, poloxamer 331, poloxamer 338, poloxamer 407 or a mixture thereof, more preferably polysorbate 20, polysorbate 80, poloxamer 188 or a mixture thereof.

The pharmaceutical composition disclosed herein comprises a pharmaceutically acceptable preservative. Suitable pharmaceutically acceptable preservatives may be selected from the group consisting of phenol, o-cresol, m-cresol, p-cresol, methyl p-hydroxybenzoate, propyl p-hydroxybenzoate, 2-phenoxyethanol, p-hydroxybenzene Butyl formate, 2-phenylethanol, benzyl alcohol, chlorobutanol, chlorocresol, ethyl p-hydroxybenzoate and a mixture thereof at a concentration in the range of about 0.1 mg/ml to 10 mg/ml, preferably at a concentration in the range of about 1 mg/ml to 8 mg/ml, and most preferably at a concentration in the range of about 2 mg/ml to 6 mg/ml.

The present invention also discloses a preparation method of a GLP-1 analog pharmaceutical composition for injection comprising the steps of:

(1) dissolving a preservative, xylitol and a buffering agent in water to prepare a solution;

(2) dissolving the GLP-1 analogous in the above solution and adjusting to the desired pH range;

(3) adding a stabilizer to the above solution to obtain the pharmaceutical formulation comprising GLP-1 analog in a concentration from 0.1 mg/ml to 25 mg/ml, a buffer with pH from 7.5 to 9.0, a stabilizer in a concentration from 0.001% to 0.5% (m/v), xylitol in a concentration from 0.5% to 10%(m/v), and preservative in a concentration from 0.1 mg/ml to 10 mg/ml.

Wherein the definition of preservatives, buffers, stabilizers and GLP-1 analog is as previously described.

Following significant experimentation, the inventors have surprisingly found that the pharmaceutical compositions disclosed herein are particularly suitable for long-term storage and preservation of pharmaceutically useful liraglutide formulations.

Liraglutide is administered subcutaneously by injection; the inventor has found that it is necessary to add a suitable amount of a surfactant to the formulation solution to stabilize the liraglutide preparation and avoid crystallization and precipitation. Clinical safety and patient tolerability limit the type and scope of surfactants that may be used. Accordingly, there is a need in the art to provide pharmaceutical compositions that improve protein stability, but contain only surfactants and other components that are compatible with human pharmaceutic use.

The present invention discloses that xylitol as an isotonic regulator (which replaces propylene glycol used in the prior art) and the combination of low concentrations of poloxamer surfactants with polysorbate surfactants (preferably Tween-20 or Tween-80) significantly improves the long-term stability of liraglutide preparation. Such improved formulations, when stored at 4° C. for 3 months, maintain purity at greater than 95%, keeps the solution clear, with no visible crystallization, precipitation, or other optically detectable changes. Further, xylitol is regarded by the diabetes clinical studies field as safer than propylene glycol with a lower potential for adverse effects including injection site reactions.

More importantly, the inventors have surprisingly found that xylitol combined with low concentrations of poloxamer surfactant and polysorbate surfactant reduces the formation of high molecular weight impurities, such as dimers, during long-term storage of liraglutide formulations. This unexpected effect has never been reported in the prior art.

Xylitol has a very low glycemic index; consumption has a negligible effect on circulating serum glucose and insulin production. Xylitol's metabolism does not depend on insulin and xylitol does not increase blood glucose levels. Xylitol can reduce the intensity or frequency of common diabetic symptoms such as polydipsia, polyuria and food cravings. Because xylitol is perceived as sweet yet has no meaningful impact on serum glucose levels and is very low calorie, it is widely used as a food additive.

In addition, xylitol is very chemical stable. It does not interact with drugs or other common excipients and can be used over a very wide pH range (pH 1-11). Second, xylitol has a lower activity and a higher osmotic pressure in the water, thus increasing the stability of the product. The art recognized that xylitol is not only a stabilizer but may have a synergistic effect with preservatives intended to protect formulations from bacterial contamination. Xylitol may enhance the bacteriostatic or bactericidal properties of common preservatives.

The invention further discloses a method for preparation of a pharmaceutical formulation for injection, which comprises the following steps:

(1) dissolving a preservative, xylitol and a buffering agent in water to prepare a solution;

(2) dissolving the liraglutide in the above solution, adjusting the pH to the desired pH;

(3) adding a stabilizer to the above solution to obtain the pharmaceutical formulation comprising liraglutide in a concentration from 0.1 mg/ml to 25 mg/ml, a buffer with pH from 7.5 to 9.0, a stabilizer in a concentration from 0.001% to 0.5% (m/v), xylitol in a concentration from 0.5% to 10%(m/v), and preservative in a concentration from 0.1 mg/ml to 10 mg/ml.

The pharmaceutical formulation prepared by the present invention comprising liraglutide in a concentration from 0.1 mg/ml to 25 mg/ml, a buffer has pH from 7.5 to 9.0, a stabilizer in a concentration from 0.001% to 0.5% (m/v), xylitol in a concentration from 0.5% to 10%(m/v), and preservative in a concentration from 0.1 mg/ml to 10 mg/ml.

The present invention has the advantage of providing a formulation suitable for multiple clinical injections by using safe and well tolerated additives to stabilize the physicochemical properties and preserve biological activity of liraglutide. This preparation effectively prevents formation of high molecular peptide polymers; prevents crystallization and precipitation; and, protects against oxidation and mechanical disturbance. Together these features yield a pharmaceutical formulation that is suitable for long term storage and distribution in the pharmaceutical supply chain.

In the pharmaceutical compositions disclosed in the present invention, the concentration of the liraglutide is preferably about 1 mg/ml to 15 mg/ml, more preferably 3 mg/ml to 10 mg/ml, and most preferably 6 mg/ml.

In the pharmaceutical composition disclosed in the present invention, a buffer suitable for use in the present invention is any buffer capable of maintaining the pH of the formulation in the aqueous solution at a pH of 7.5 to 9.0, which can be selected from the group consisting of phosphate buffer, a disodium hydrogen phosphate-citrate buffer, TRIS buffer, glycyl-glycine buffer, N-bis (hydroxyethyl) glycine buffer, sodium dihydrogen phosphate buffer, disodium hydrogen phosphate buffer, sodium acetate buffer, sodium carbonate buffer, sodium phosphate buffer, lysine buffer, arginine buffer and a mixture thereof.

The pH of the buffer is preferably in the range of 7.5 to 8.5, more preferably 8.0 to 8.5; the concentration of the buffer is 5 to 100 mmol/L, preferably 10 to 30 mmol/L. Preferably the buffer is the disodium hydrogen phosphate buffer which concentration is in the range of 5 to 100 mmol/L and the pH is in the range of 7.5 to 8.5; more preferably the concentration is in the range of 10 to 30 mmol/L and the pH is in the range of 8.0 to 8.5.

The said pharmaceutical composition uses xylitol as an isotonic regulator in a concentration range from 0.5% to 10% (m/v), more preferably 1% to 5% (m/v). Chinese patent application CN200480034152.8 filed by Novo Nordisk, teaches away from commonly used isotonic regulators such as glucose, mannitol, xylitol, fructose, lactose, maltose, sucrose, trehalose, glycerol, glycine, histidine or arginine. That art teaches that such isotonic regulators cannot be used with liraglutide because they stimulate liraglutide crystallization from the solution.

Here, the inventors disclose that xylitol used in combination with surfactants promote stable liraglutide solutions and prevent crystallization over a broad range of conditions. Further, the inventors have surprisingly found that xylitol reduces formation of high molecular weight impurities, such as dimers, in liraglutide formulations. These stabilized formulations are more stable than the currently marketed Victoza formulations. This unexpected effect of xylitol and the resulting properties of the formulation has never reported in the relevant art.

In the pharmaceutical compositions disclosed, a stabilizer for improving the stability of liraglutide is preferably selected from the group consisting of polysorbate 20, polysorbate 80, poloxamer 124, poloxamer 181, poloxamer 182, poloxamer 188, poloxamer 237, poloxamer 331, poloxamer 338, poloxamer 407 and a mixture thereof, more preferably selected from the group consisting of polysorbate 20, polysorbate 80, poloxamer 188 and a mixture thereof.

The pharmaceutical compositions disclosed comprises a pharmaceutically acceptable preservative. Suitable pharmaceutically acceptable preservatives may be selected from the group consisting of phenol, o-cresol, m-cresol, p-cresol, methyl p-hydroxybenzoate, propyl p-hydroxybenzoate, 2-phenoxyethanol, p-hydroxybenzene butyl formate, 2-phenylethanol, benzyl alcohol, chlorobutanol, chlorocresol, ethyl p-hydroxybenzoate and a mixture thereof in a concentration from about 0.1 mg/ml to 10 mg/ml, preferably in a concentration from about 1 mg/ml to 8 mg/ml, and most preferably in a concentration from about 2 mg/ml to 6 mg/ml.

The preferred preservative in the pharmaceutical compositions are phenol or m-cresol, which may be used alone or in combination. A more preferred preservative is phenol at a concentration in the range of about 0.1 mg/ml to about 10 mg/ml, preferably at a concentration from about 1 mg/ml to 8 mg/ml, and most preferably at a concentration of about 2 mg/ml to 6 mg/ml.

The inventors found that a stabilizer, particularly polysorbate 20, polysorbate 80, poloxamer 188 or a mixture thereof, at a concentration in the range of 0.001 to 0.5% (m/v) combined with xylitol effectively promote the long-term stability of liraglutide liquid formulations and prevent liraglutide precipitation and crystallization. Polysorbate 20 or polysorbate 80 and poloxamer 188 may be used alone or in combination. When used alone, polysorbate 20, polysorbate 80 or poloxamer 188 concentration should preferably be from about 0.004% to about 0.3%(m/v), most preferably 0.02%(m/v). When used in combination, the concentration of polysorbate 80should be from about 0.001% to about 0.3% (m/v) and the concentration of poloxamer 188should be from about 0.001% to about 0.3% (m/v). More preferably the concentration of polysorbate 80 should be from about 0.004% to about 0.2% (m/v) and the concentration of poloxamer 188 should be from about 0.004% to about 0.2%(m/v); more preferably the concentration of polysorbate 80 is 0.01% (m/v) and the concentration of poloxamer 188 is 0.01%(m/v). The combination of a stabilizer and xylitol is particularly useful for preventing liraglutide precipitation from the solution and for inhibiting formation of high molecular impurities, such as dimers.

Preferably, the above pharmaceutical compositions comprise liraglutide at a concentration from 0.1 mg/ml to 25 mg/ml, pH from 7.5 to 9.0, disodium hydrogen phosphate buffer at a concentration from 5 mmol/L to 100 mmol/L, polysorbate 80 or poloxamer 188 at a concentration from 0.001% to 0.5% (m/v), xylitol at a concentration from 0.5% to 10% (m/v),and phenol or m-cresol at a concentration from 0.1 mg/ml to 10 mg/ml.

Preferably, the above pharmaceutical composition comprises liraglutide at a concentration from 0.1 mg/ml to 25 mg/ml, pH from 7.5 to 9.0, disodium hydrogen phosphate buffer at a concentration from 5 mmol/L to 100 mmol/L, polysorbate 80 at a concentration from 0.001% to 0.5% (m/v), poloxamer 188 at a concentration from 0.001% to 0.5%(m/v), xylitol at a concentration from 0.5% to 10% (m/v), and phenol or m-cresol at a concentration from 0.1 mg/ml to 10 mg/ml.

Preferably, the above pharmaceutical composition comprises liraglutide at a concentration from 1 mg/ml to 15 mg/ml, pH from 7.5 to 8.5, disodium hydrogen phosphate buffer at a concentration from 10 mmol/L to 30 mmol/L, polysorbate 80 or poloxamer 188 at a concentration from 0.004% to 0.3% (m/v), xylitol at a concentration from 1% to 5% (m/v), and phenol or m-cresol at a concentration from 1 mg/ml to 8 mg/ml.

Preferably, the above pharmaceutical composition comprises liraglutide at a concentration from 1 mg/ml to 15 mg/ml, pH from 7.5 to 8.5, disodium hydrogen phosphate buffer at a concentration from 10 mmol/L to 30 mmol/L, polysorbate 80 at a concentration from 0.004% to 0.3% (m/v), poloxamer 188 at a concentration from 0.004% to 0.3% (m/v), xylitol at a concentration from 1% to 5% (m/v), and phenol or m-cresol at a concentration from 1 mg/ml to 8mg/ml.

Preferably, the above pharmaceutical composition comprises liraglutide at a concentration from 1 mg/ml to 15mg/ml, pH from 7.5 to 8.5, disodium hydrogen phosphate buffer at a concentration from 10 mmol/L to 30 mmol/L, polysorbate 80 at a concentration from 0.004% to 0.3% (m/v), poloxamer 188 at a concentration from 0.004% to 0.3% (m/v), xylitol at a concentration froml % to 5% (m/v), phenol or m-cresol at a concentration from 1 mg/ml to 8 mg/ml.

Preferably, the above pharmaceutical composition comprises liraglutide at a concentration from 3 mg/ml to 10 mg/ml, pH from 8.0 to 8.5, disodium hydrogen phosphate buffer at a concentration from 10 mmol/L to 30 mmol/L, polysorbate 80 or poloxamer 188 at a concentration 0.02% (m/v), xylitol at a concentration from 1% to 5% (m/v), phenol or m-cresol at a concentration from 2 mg/ml to 6 mg/ml.

Preferably, the above pharmaceutical composition comprises liraglutide at a concentration from 3 mg/ml to 10 mg/ml, pH from 8.0 to 8.5, disodium hydrogen phosphate buffer at a concentration from 10 mmol/L to 30 mmol/L, polysorbate 80 at a concentration 0.01%(m/v), poloxamer 188 at a concentration 0.01% (m/v), xylitol at a concentration from 1% to 5% (m/v), and phenol or m-cresol at a concentration from 2 mg/ml to 6 mg/ml.

Preferably, the above method wherein stabilizer in method step(3) is a mixture of polysorbate 80 and poloxamer 188, comprising polysorbate 80 in a concentration from about 0.004% to about 0.3%(m/v) and poloxamer 188 in a concentration from about 0.004% to about 0.3%(m/v). Most preferably, the above method, wherein the stabilizer in method step(3) is a mixture of polysorbate 80 and poloxamer 188, comprising polysorbate 80 in a concentration 0.01%(m/v) and poloxamer 188 in a concentration 0.01%(m/v).

The above disclosed pharmaceutical compositions can be prepared as a lyophilized powder; the lyophilized powder can be reconstituted using a pharmaceutically acceptable diluent, for example, by adding water for injection. Lyophilization can be carried out using common techniques in the art, for example, freeze-drying cycles that include freezing, primary drying and secondary drying. Because the liquid preparations before lyophilization are substantially isotonic and/or iso-osmolar, the lyophilized powder can be reconstituted back to anisotonic or iso-osmolar solution by simply adding an appropriate amount of water for injection.

The above pharmaceutical compositions are useful for the treatment of patients with type 2 diabetes mellitus, for improving blood glucose control, either alone or in combination with metformin, sulfonylureas or similar drugs. The pharmaceutical composition is administrated by subcutaneous injection, with one of the preferred dosing regimens being once daily with an escalating regimen wherein 0.6 mg/day is administered for the first week and 1.2 mg/day in the weeks thereafter. If the 1.2 mg dose does not significantly control blood glucose, the dose is increased to 1.8 mg/day.

These disclosed formulations may be used to treat patients with type 2 diabetes mellitus. Further, these formulations can be administered alone or in combination with metformin, sulfonylureas or similar therapeutics. In a preferred embodiment, a disclosed formulation may be administered as a subcutaneous injection. To enhance patient tolerance, a preferred dosing regimen is once daily, 0.6 mg/day for the first week and 1.2 mg/day or 1.8 mg/day thereafter.

The said method for preparing a liraglutide pharmaceutical composition, wherein the preservative, the buffer and the stabilizer are defined as described above, and the preferred liraglutide pharmaceutical composition is also as described above.

The solution obtained in step (3) can be filtered with a 0.22 μm filter, and then can be used for further formulation or compounding.

The following examples are provided for clarity of the present invention, and are merely illustrative of the present invention and are not intended to be limiting.

### EXAMPLES

### Example 1

Investigating the dissolution of liraglutide powder at different pHs

The appropriate amount of liraglutide powder was dissolved in the water for injection and the disodium hydrogen phosphate buffer at a different pH was added resulting in the dissolution shown in Table 1.

TABLE 1
The dissolution of the liraglutide powder
in the solution at different pHs

| | The concentration | |
|---|---|---|
| | of liraglutide | Dissolution |
| water for injection | 6 mg/ml | insoluble |
| 10 mM disodium hydrogen phosphate | 6 mg/ml | insoluble |
| buffer (pH 7.00) | | |
| 10 mM disodium hydrogen phosphate | 6 mg/ml | colorless |
| buffer (pH 7.50) | | and clear |
| 10 mM disodium hydrogen phosphate | 6 mg/ml | colorless |
| buffer (pH 8.00) | | and clear |
| 10 mM disodium hydrogen phosphate | 6 mg/ml | colorless |
| buffer (pH 8.15) | | and clear |
| 10 mM disodium hydrogen phosphate | 6 mg/ml | colorless |
| buffer (pH 8.50) | | and clear |
| 10 mM disodium hydrogen phosphate | 6 mg/ml | colorless |
| buffer (pH 9.00) | | and clear |

From the above test we could know: liraglutide powder didn't dissolve in acidic and neutral conditions,and were readily dissolved in alkaline conditions.

### Example 2

Investigating the osmotic pressure of the solution containing different isotonic agents

The isotonic agent was dissolved in 10 mM disodium phosphate buffer and liraglutide was added to 6 mg/ml with stirring and the pH was adjusted to pH 8.15 with sodium hydroxide. Finally, the solution was filtered through a 0.22 μm filter. The concentration of each solution isotonic agent and osmotic pressure test results shown in Table 2.

TABLE 2
Concentration of isotonic agent and osmotic pressure test results

| Isotonic agent | Osmotic pressure |
|---|---|
| Negative control (no isotonic agent) | 0.041 |
| Methionine (15 mg/ml) | 0.141 |
| Glycine (15 mg/ml) | 0.301 |
| Xylitol (28 mg/ml) | 0.284 |
| PEG 400 (61 mg/ml) | 0.291 |
| L-arginine (25 mg/ml) | 0.322 |
| Sorbitol (32 mg/ml) | 0.277 |
| Glycerol (16.8 mg/ml) | 0.289 |
| Sodium chloride (8.6 mg/ml) | 0.307 |
| Victoza | 0.281 |

The isotonic solution had an osmolality of about 0.285 to 0.310 osmol/L.

### Example 3

Examining the stability of the formulation solution containing different stabilizers

The preservatives, isotonic agents and buffers were dissolved in water for injection and the liraglutide powder was dissolved in the solution with slow stirring. Then the pH was adjusted to the desired pH with sodium hydroxide and/or hydrochloric acid. Once the pH was adjusted the indicated amount of a stabilizer was added. Finally, the above formulation solution was filtered through a 0.22 μm filter. The type and amount of stabilizer added were shown in Table 3.

The composition of the formulation was as follows:

Liraglutide: 6 mg/ml

Disodium hydrogen phosphate: 1.42 mg/ml

Phenol: 5.5 mg/ml

Isotonic agent: appropriate amount

Stabilizer: appropriate amount

Water for injection: to 1 ml

pH : 8.15

TABLE 3
Type & Amount of Stabilizers

| | Isotonic | Concen- | | Concen- |
|---|---|---|---|---|
| No. | agents | tration | Stabilizer | tration |
| 1 | Xylitol | 28 mg/ml | Polysorbate 80 | 0.02% |
| 2 | Xylitol | 28 mg/ml | Poloxamer 188 | 0.02% |
| 3 | Xylitol | 28 mg/ml | Polysorbate 80 + | 0.01% + 0.01% |
| | | | Poloxamer 188 | |
| 4 | Sodium | 8 mg/ml | Polysorbate 80 + | 0.01% + 0.01% |
| | chloride | | Poloxamer 188 | |
| 5 | Xylitol | 28 mg/ml | Hydroxypropyl-β- | 2% |
| | | | cyclodextrin | |
| 6 | Xylitol | 28 mg/ml | PovidoneK 30 | 3% |
| 7 | Xylitol | 28 mg/ml | PEG 300 | 3% |
| 8 | — | — | Victoza prescription | 14 mg/ml |
| | | | (including propylene | |
| | | | glycol) | |

The above preparations were incubated at 37° C., 25° C. and 4° C. for stability investigation, and the related substances (area normalization) of the samples were detected by HPLC. The results are shown below in Tables 4, 5, and 6.

TABLE 4
Stability test at 37° C.

| | Formulation No. | | | | | | | |
|---|---|---|---|---|---|---|---|---|
| Time | 1 | 2 | 3 | 4 | 5 | 6 | 7 | 8 |
| Start time | 0.17% | 0.18% | 0.20% | 0.18% | 2.48% | 0.14% | 0.18% | 0.50% |
| 1 week | 1.38% | 0.70% | 1.66% | 1.63% | 2.59% | 27.6% | 4.24% | 1.58% |
| 2 week | 1.52% | 1.47% | 2.03% | 2.13% | 6.00% | 69.31% | 5.15% | 1.72% |
| 3 week | 3.77% | 3.85% | 4.56% | 4.46% | 7.41% | — | 6.73% | 4.73% |
| 4 week | 3.51% | 3.33% | 4.18% | 4.63% | 9.23% | — | 6.34% | 4.29% |

TABLE 5
Stability test at 25° C.

| | Formulation No. | | | | | | | |
|---|---|---|---|---|---|---|---|---|
| Time | 1 | 2 | 3 | 4 | 5 | 6 | 7 | 8 |
| Start time | 0.17% | 0.18% | 0.20% | 0.18% | 2.48% | 0.14% | 0.18% | 0.50% |
| 1 week | 0.84% | 0.80% | 0.85% | 0.91% | 3.19% | 21.16% | 2.41% | 2.63% |
| 2 week | 1.33% | 1.20% | 1.99% | 1.99% | 3.82% | 64.26% | 4.29% | 1.99% |
| 3 week | 1.92% | 2.07% | 1.94% | 2.12% | 5.04% | — | 4.97% | 2.22% |
| 4 week | 2.22% | 2.19% | 2.32% | 2.57% | 6.77% | — | 5.50% | 2.76% |

TABLE 6
Stability test at 4° C.

| | Formulation No. | | | | | | | |
|---|---|---|---|---|---|---|---|---|
| Time | 1 | 2 | 3 | 4 | 5 | 6 | 7 | 8 |
| Start time | 0.17% | 0.18% | 0.20% | 0.18% | 2.48% | 0.14% | 0.18% | 0.50% |
| 2 week | 0.31% | 0.48% | 0.68% | 0.59% | 3.05% | 16.84% | 1.53% | 1.02% |
| 4 week | 0.49% | 0.80% | 1.18% | 1.11% | 3.55% | — | 2.15% | 1.69% |

From the above results we see that low concentration of polysorbate 80 or poloxamer 188 can effectively increase the stability of liraglutide preparation. During the stability study period preparations 1 through 4 were clear with no visible precipitation or crystallization observed. Even in the accelerated stability tests the formulations maintain the properties of the preparation more than 95%, showing its superiority to hydroxypropyl-β-cyclodextrin, povidoneK30 or PEG 300. The observed stability of the disclosed formulations is better than the stability of the currently marketed Victoza formulation.

### Example 4

Investigating formulation stability with different concentrations with poloxamer 188 used as the stabilizer

The preservatives, isotonic agents and buffers are dissolved in water for injection, then liraglutide powder was dissolved in the solution with slow stirring. Next, the pH was adjusted to the desired pH with sodium hydroxide and/or hydrochloric acid. Then the indicated amount of poloxamer 188 was added. Finally, the formulation solution was filtered through a 0.22 μm filter. The amount of poloxamer 188 added is shown below in Table 7.

The composition of the preparation was as follows:
Liraglutide: 6 mg/ml
Disodium hydrogen phosphate: 1.42 mg/ml
Phenol: 5.5 mg/ml
Xylitol: 28.0 mg/ml
Poloxamer 188: as indicated in Table 7
Water for injection: to 1 ml
pH: 8.15

TABLE 7
poloxamer 188 concentration of different prescriptions

| Formulation No. | Poloxamer 188 Amount |
|---|---|
| 1 | Victoza formulation |
| 2 | 0 |
| 3 | 0.004% |
| 4 | 0.01% |
| 5 | 0.02% |
| 6 | 0.03% |
| 7 | 0.04% |
| 8 | 0.05% |
| 9 | 0.1% |
| 10 | 0.2% |

The above preparations were incubated at 37° C., 25° C. and 4° C. for accelerated stability analysis. Following incubation at the indicated temperature, the composition of each sample was determined by reverse phase HPLC (area normalization). SEC HPLC (area normalization) was used to detect high molecular weight impurities such as polymers and/or dimers. During the stability study, each solution was clear with visible foreign matter, precipitation or crystallization observed. Tables 8-13 below show the results of the reverse phase and SEC HPLC analysis.

TABLE 8
The HPLC related substances results of stability test at 37° C.

Time

| No. | Start time | 1 month | 2 month | 3 month | 6 month |
|---|---|---|---|---|---|
| 1 | 0.88% | 4.06% | 15.78% | 22.38% | 31.56% |
| 2 | 0.92% | 5.71% | 19.74% | 19.39% | 31.67% |
| 3 | 0.92% | 3.32% | 11.05% | 20.27% | 33.88% |
| 4 | 0.92% | 3.28% | 10.96% | 19.37% | 32.06% |
| 5 | 0.90% | 3.46% | 11.09% | 18.96% | 32.67% |
| 6 | 0.89% | 3.56% | 11.62% | 19.67% | 34.22% |
| 7 | 0.88% | 3.49% | 11.53% | 20.32% | 34.55% |
| 8 | 0.91% | 3.58% | 12.79% | 20.52% | 35.17% |
| 9 | 0.88% | 3.75% | 12.15% | 20.90% | 33.73% |
| 10 | 0.91% | 3.61% | 11.70% | 20.97% | 33.69% |

TABLE 9
The HPLC related substances results of stability test at 25° C.

Time

| No. | Start time | 1 month | 2 month | 3 month | 6 month |
|---|---|---|---|---|---|
| 1 | 0.88% | 1.52% | 3.24% | 4.63% | 6.39% |
| 2 | 0.92% | 3.42% | 5.82% | 3.53% | 6.39% |
| 3 | 0.92% | 1.43% | 3.01% | 3.83% | 6.66% |
| 4 | 0.92% | 1.45% | 2.93% | 3.83% | 6.73% |
| 5 | 0.90% | 1.46% | 2.56% | 3.87% | 6.58% |
| 6 | 0.89% | 1.51% | 2.60% | 3.79% | 6.47% |
| 7 | 0.88% | 1.50% | 2.95% | 3.95% | 6.60% |
| 8 | 0.91% | 1.55% | 3.06% | 4.08% | 6.69% |
| 9 | 0.88% | 2.43% | 3.03% | 4.14% | 6.78% |
| 10 | 0.91% | 2.50% | 3.11% | 4.13% | 7.04% |

TABLE 10
The HPLC related substances results of stability test at 4° C.

| | Time | | | | |
|---|---|---|---|---|---|
| No. | Start time | 3 month | 6 month | 9 month | 12 month |
| 1 | 0.88% | 1.33% | 2.11% | 1.28% | 3.17% |
| 2 | 0.92% | 1.63% | 1.10% | 1.20% | 2.96% |
| 3 | 0.92% | 1.09% | 1.11% | 1.02% | 2.81% |
| 4 | 0.92% | 1.09% | 1.15% | 0.99% | 2.74% |
| 5 | 0.90% | 1.03% | 1.07% | 0.97% | 3.07% |
| 6 | 0.89% | 1.04% | 1.05% | 0.95% | 2.97% |
| 7 | 0.88% | 1.04% | 1.06% | 0.96% | 3.21% |
| 8 | 0.91% | 1.05% | 1.08% | 0.96% | 2.77% |
| 9 | 0.88% | 0.98% | 1.08% | 0.95% | 2.86% |
| 10 | 0.91% | 1.14% | 1.13% | 1.00% | 3.35% |

TABLE 11
SEC HPLC High molecular impurities
results of stability test at 37° C.

Time

| No. | Start time | 1 month | 2 month | 3 month | 6 month |
|---|---|---|---|---|---|
| 1 | 0.45% | 1.01% | 3.70% | 4.93% | 8.25% |
| 2 | 0.09% | 1.84% | 4.50% | 4.81% | 8.69% |
| 3 | 0.11% | 0.89% | 2.60% | 4.88% | 8.72% |
| 4 | 0.09% | 0.92% | 3.05% | 4.87% | 8.57% |
| 5 | 0.10% | 0.95% | 2.68% | 4.65% | 9.66% |
| 6 | 0.13% | 0.97% | 2.90% | 4.67% | 9.53% |
| 7 | 0.11% | 0.84% | 2.63% | 4.80% | 10.53% |
| 8 | 0.12% | 0.89% | 2.70% | 4.83% | 9.04% |
| 9 | 0.12% | 1.04% | 2.86% | 4.31% | 9.60% |
| 10 | 0.13% | 1.01% | 2.54% | 4.63% | 9.57% |

TABLE 12
SEC HPLC High molecular impurities
results of stability test at 25° C.

Time

| No. | Start time | 1 month | 2 month | 3 month | 6 month |
|---|---|---|---|---|---|
| 1 | 0.45% | 0.61% | 1.04% | 1.34% | 2.10% |
| 2 | 0.09% | 0.90% | 1.51% | 1.08% | 2.22% |
| 3 | 0.11% | 0.49% | 0.83% | 1.15% | 1.95% |
| 4 | 0.09% | 0.42% | 0.88% | 1.09% | 2.06% |
| 5 | 0.10% | 0.57% | 0.81% | 1.08% | 1.90% |
| 6 | 0.13% | 0.57% | 0.87% | 1.11% | 1.95% |
| 7 | 0.11% | 0.50% | 0.86% | 1.12% | 2.07% |
| 8 | 0.12% | 0.62% | 0.90% | 1.23% | 2.06% |
| 9 | 0.12% | 0.54% | 0.80% | 1.18% | 2.08% |
| 10 | 0.13% | 0.61% | 0.88% | 1.23% | 2.05% |

TABLE 13
SEC HPLC High molecular impurities
results of stability test at 4° C.

| | Time | | | | |
|---|---|---|---|---|---|
| No. | Start time | 3 month | 6 month | 9 month | 12 month |
| 1 | 0.45% | 0.32% | 0.37% | 0.56% | 1.30% |
| 2 | 0.09% | 0.24% | 0.35% | 0.50% | 1.40% |
| 3 | 0.11% | 0.30% | 0.26% | 0.55% | 0.87% |
| 4 | 0.09% | 0.30% | 0.38% | 0.50% | 1.08% |
| 5 | 0.10% | 0.30% | 0.38% | 0.54% | 1.10% |
| 6 | 0.13% | 0.35% | 0.35% | 0.56% | 1.14% |
| 7 | 0.11% | 0.31% | 0.41% | 0.53% | 1.35% |
| 8 | 0.12% | 0.32% | 0.33% | 0.56% | 1.13% |
| 9 | 0.12% | 0.28% | 0.33% | 0.49% | 1.23% |
| 10 | 0.13% | 0.39% | 0.32% | 0.60% | 1.30% |

From the above results we determined that the physical stability and chemical stability of the liraglutide preparation containing 0.004% -0.03% poloxamer 188 and isotonic agent xylitol were significantly increased. When stored at 4° C. for 12 months, the purity of liraglutide remained greater than 97%; further, the solution remained clear, with no visible foreign matter, crystallization, or precipitation. The disclosed formulations show much reduced levels of high molecular weight impurities as compared to the presently marketed Victoza formulation.

1-82. (canceled)

83. A pharmaceutical formulation comprising active ingredient liraglutide at a concentration from 0.1 mg/ml to 25 mg/ml, disodium hydrogen phosphate buffer at a concentration from 5 mmol/L to 100 mmol/L, polysorbate 80 or poloxamer 188 at a concentration from 0.001% to 0.5% (m/v), poloxamer 188 at a concentration from 0.001% to 0.5% (m/v), xylitol at a concentration from 0.5% to 10% (m/v), phenol or m-cresol at a concentration from 0.1 mg/ml to 10 mg/ml, wherein the formulation has a pH from 7.5 to 9.0.

84. A pharmaceutical formulation according to claim 83, comprising polysorbate 80 at a concentration from 0.001% to 0.5% (m/v).

85. A pharmaceutical formulation according to claim 84, wherein said liraglutide is present at a concentration from 3 mg/ml to 10 mg/ml, said disodium hydrogen phosphate buffer at a concentration from 10 mmol/L to 30 mmol/L, said polysorbate 80 at a concentration from 0.004% to 0.3% (m/v), poloxamer 188 at a concentration from 0.004% to 0.3% (m/v), said xylitol at a concentration from 1% to 5% (m/v), said phenol or m-cresol at a concentration from 2 mg/ml to 6 mg/ml, wherein said formulation has a pH from 7.5 to 8.5.

86. A pharmaceutical formulation comprising a GLP-1 analog in a concentration from 0.1 mg/ml to 25 mg/ml, a buffer at a pH from 7.5 to 9.0, a stabilizer in a concentration from 0.001% to 0.5% (m/v), xylitol in a concentration from 0.5% to 10% (m/v), and a preservative at a concentration from 0.1 mg/ml to 10 mg/ml;
wherein the GLP-1 analog is selected from the group consisting of GLP-1, GLP-1 (7-36)-amide, GLP-1 (7-37), and GLP-1 derivatives.

87. The formulation according to claim 86, wherein the GLP-1 derivative has a lipophilic substituent attached, wherein the lipophilic substituent has 4-40 carbon atoms, 8-30 carbon atoms, 8-25 carbon atoms, 12-25 carbon atoms, or 14-18 carbon atoms.

88. The formulation according to claim 86, wherein the GLP-1 analog is Arg³⁴Lys²⁶(N^{ε}-(γ-Glu(N^{α}-hexadecanoyl)))-GLP-1(7-37).

89. The formulation according to claim 86, wherein the GLP-1 analog is selected from the group consisting of Gly⁸-GLP-1(7-36)-amide,Gly⁸-GLP-1(7-37), Val⁸-GLP-1(7-36)-amide, Val⁸-GLP-1(7-37), Val⁸Asp²²-GLP-1(7-37), Val⁸Asp²²-GLP-1(7-36)-amide, Val⁸Glu²²-GLP-1 (7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), Arg²⁶-GLP-1(7-37), Arg³⁴-GLP-1(7-37), Lys³⁶-GLP-1 (7-37), Arg^{26 34}Lys³⁶-GLP-1(7-37), Arg^{26 34}-GLP-1(7-37), Arg^{26,34}Lys⁴⁰-GLP-1(7-37), Arg²⁶Lys³⁶-GLP-1(7-37), Arg³⁴Lys³⁶-GLP-1 (7-37), Val⁸Arg²²-GLP-1(7-37), Met⁸Arg²²-GLP-1(7-37), Gly⁸His²²-GLP-1 (7-37), Val⁸His²²-GLP-1(7-37), Met⁸His²²-GLP-1(7-37), His³⁷-GLP-1(7-37), Gly⁸-GLP-1(7-37), Val⁸-GLP-1(7-37), Met⁸-GLP-1(7-37), Gly⁸Asp²²-GLP-1(7-37), Val⁸Asp²²-GLP-1(7-37), Met⁸Asp²²-GLP-1(7-37), Gly⁸Glu²²-GLP-1 (7-37), Val⁸Glu²²-GLP-1(7-37), Met⁸Glu²²-GLP-1(7-37), Gly⁸Lys²²-GLP-1(7-37), Val⁸Lys²²-GLP-1(7-37), Met⁸Lys²²-GLP-1(7-37), Gly⁸Arg²²-GLP-1(7-37), Val⁸Lys²²His³⁷-GLP-1 (7-37), Gly⁸Glu²²His³⁷-GLP-1(7-37), Val⁸Glu²²His³⁷-GLP-1(7-37), Met⁸Glu²²His³⁷-GLP-1(7-37), Gly⁸Lys²²His³⁷-GLP-1(7-37), Met⁸Lys²²His³⁷-GLP-1(7-37), Gly⁸Arg²²His³⁷-GLP-1(7-37), Val⁸Arg²²His³⁷-GLP-1(7-37), Met⁸Arg²²His³⁷-GLP-1(7-37), Gly⁸His²²His³⁷-GLP-1(7-37), Val⁸His²²His³⁷-GLP-1(7-37), Met⁸His³³His³⁷-GLP-1 (7-37), Gly⁸His³⁷-GLP-1(7-37), Val⁸His³⁷-GLP-1(7-37), Met⁸His³⁷-GLP-1(7-37), Gly⁸Asp²²His³⁷-GLP-1(7-37), Val⁸Asp²²His³⁷-GLP-1(7-37), Met⁸Asp²²His³⁷-GLP-1(7-37), Arg²⁶-GLP-1(7-36)-amide, Arg³⁴-GLP-1(7-36)-amide, Lys³⁶-GLP-1(7-36)-amide, Arg^{26,34}Lys³⁶-GLP-1(7-36)-amide, Arg^{26,34}-GLP-1(7-36)-amide, Arg^{26,34}Lys⁴⁰-GLP-1(7-36)-amide, Arg²⁶Lys³⁶-GLP-1(7-36)-amide, Arg³⁴Lys³⁶-GLP-1(7-36)-amide, Gly⁸-GLP-1(7-36)-amide, Val⁸-GLP-1(7-36)-amide, Met⁸-GLP-1(7-36)-amide, Gly⁸Asp²²-GLP-1(7-36)-amide, Gly⁸Glu²²His³⁷-GLP-1(7-36)-amide, Val⁸Asp²²-GLP-1(7-36)-amide, Met⁸Asp²²-GLP-1(7-36)-amide, Gly⁸Glu²²-GLP-1(7-36)-amide, Val⁸Glu²²-GLP-1(7-36)-amide, Met⁸Glu²²-GLP-1(7-36)-amide, Gly⁸Lys²²-GLP-1(7-36)-amide, Val⁸Lys²²-GLP-1(7-36)-amide, Met⁸Lys²²-GLP-1(7-36)-amide, Gly⁸His²²His³⁷-GLP-1(7-36)-amide, Gly⁸Arg²²-GLP-1(7-36)-amide, Val⁸Arg²²-GLP-1(7-36)-amide, Met⁸Arg²²-GLP-1(7-36)-amide, Gly⁸His²²-GLP-1(7-36)-amide, Val⁸His²²-GLP-1(7-36)-amide, Met⁸His²²-GLP-1(7-36)-amide, His³⁷-GLP-1(7-36)-amide, Val⁸Arg²²His³⁷-GLP-1(7-36)-amide, Met⁸Arg²²His³⁷-GLP-1(7-36)-amide, Gly⁸His³⁷-GLP-1(7-36)-amide, Val⁸His³⁷-GLP-1(7-36)-amide, Met⁸His³⁷-GLP-1(7-36)-amide, Gly⁸Asp²²His³⁷-GLP-1(7-36)-amide, Val⁸Asp²²His³⁷-GLP-1(7-36)-amide, Met⁸Asp²²His³⁷-GLP-1(7-36)-amide, Val⁸Glu²²His³⁷-GLP-1(7-36)-amide, Met⁸Glu²²His³⁷-GLP-1(7-36)-amide, Gly⁸Lys²²His³⁷-GLP-1(7-36)-amide, Val⁸Lys²²His³⁷-GLP-1(7-36)-amide, Met⁸Lys²²His³⁷-GLP-1(7-36)-amide, Gly⁸Arg²²His³⁷-GLP-1(7-36)-amide, Val⁸His²²His³⁷-GLP-1(7-36)-amide, Met⁸His²²His³⁷-GLP-1(7-36)-amide, Val⁸Trp¹⁹Glu²²-GLP-1(7-37), Val⁸Glu²²Val²⁵-GLP-1(7-37), Val⁸Tyr¹⁶Glu²²-GLP-1(7-37), Val⁸Trp¹⁶Glu²²-GLP-1(7-37), Val⁸Leu¹⁶Glu²²-GLP-1(7-37), Val⁸Tyr¹⁸Glu²²-GLP-1(7-37), Val⁸Glu²²His³⁷GLP-1(7-37), Val⁸Glu²²Ile³³-GLP-1(7-37), Val⁸Trp¹⁶Glu²²Val²⁵Ile³³-GLP-1(7-37), Val⁸Trp¹⁶Glu²²Ile³³-GLP-1(7-37), Val⁸Glu²²Val²⁵Ile³³-GLP-1(7-37), and Val⁸Trp¹⁶Glu²²Val²⁵-GLP-1(7-37).

90. The formulation according to claim 86, wherein the concentration of the GLP-1 analog is from about 1 mg/ml to 15 mg/ml, from 3 mg/ml to 10 mg/ml, or 6 mg/ml.

91. The formulation according to claim 86, wherein said buffer is selected from the group consisting of phosphate buffer, a disodium hydrogen phosphate-citrate buffer, TRIS buffer, glycyl-glycine buffer, N-bis (hydroxyethyl) glycine buffer, sodium dihydrogen phosphate buffer, disodium hydrogen phosphate buffer, sodium acetate buffer, sodium carbonate buffer, sodium phosphate buffer, lysine buffer, arginine buffer and mixtures thereof.

92. The formulation according to claim 91, wherein the pH of said buffer is from 7.5 to 8.5 or from 8.0 to 8.5.

93. The formulation according to claim 91, wherein the concentration of said buffer is from 5 mmol/L to 100 mmol/L or from 10 mmol/L to 30 mmol/L.

94. The formulation according to claim 93, wherein the buffer is disodium hydrogen phosphate buffer at a concentration from 5 mmol/L to 100 mmol/L, and the pH is in the range of 7.5 to 8.5.

95. The formulation according to claim 86, wherein said xylitol is present in a concentration from 1% to 5% (m/v).

96. The formulation according to claim 86, wherein the stabilizer is selected from the group consisting of glycine, alanine, serine, aspartic acid, glutamic acid, threonine, tryptophan, lysine, hydroxy lysine, histidine, arginine, cystine, cysteine, methionine, phenylalanine, leucine, isoleucine amino acids and their derivatives, sorbitan fatty acid esters, glycerol fatty acid esters (e.g., sorbitan monoate, sorbitan monolaurate and sorbitan palm Acid monoester), polyglycerol fatty acid esters (e.g., glyceryl octanoic acid monoester, glyceryl myristate mono-tallow cream and glycerol hard fatty acid monoester), polyoxyethylene sorbitan fatty acid esters, polyoxyethylene sorbitol fatty acid esters, polyoxyethylene glycerol fatty acid esters, polyoxyethylene glycol fatty acid esters, polyoxyethylene alkyl ethers, polyoxyethylene polyoxypropylene alkyl ethers, polyoxyethylene phenyl ethers, polyoxyethylated hard castor oil, polyoxyethylated beeswax derivatives, polyoxyethylenated lanolin derivatives or a polyoxyethylene fatty acid amide, alkyl sulfate, polyethylene glycol, polyvinyl alcohol, hydroxypropyl-Dextrins, carboxymethylcellulose, polyvinylpyrrolidone, polysorbate 20, polysorbate 80, poloxamer 124, poloxamer 181, poloxamer 182, poloxamer 188, poloxamer 237, poloxamer 331, poloxamer 338, and poloxamer 407.

97. The formulation according to claim 96, wherein the stabilizer is selected from the group consisting of polysorbate 20, polysorbate 80, poloxamer 124, poloxamer 181, poloxamer 182, poloxamer 188, poloxamer 237, poloxamer 331, poloxamer 338, poloxamer 407 and mixtures thereof.

98. The formulation according to claim 97, wherein a stabilizer is selected from the group consisting of polysorbate 20, polysorbate 80, poloxamer 188 and mixtures thereof.

99. The formulation according to claim 86, wherein said preservative is selected from the group consisting of phenol, o-cresol, m-cresol, p-cresol, methyl p-hydroxybenzoate, propyl p-hydroxybenzoate, 2-phenoxyethanol, p-hydroxybenzene butyl formate, 2-phenylethanol, benzyl alcohol, chlorobutanol, chlorocresol, ethyl p-hydroxybenzoate and mixtures thereof.

100. The formulation according to claim 99, wherein said preservative is present in a concentration from about 2 mg/ml to 6 mg/ml.

101. A method of treating a patient with type two diabetes mellitus comprising administering an effective amount of a formulation according to claim 83.

102. A method of preparing the pharmaceutical formulation according to claim 86 for injection, comprising the following steps:
(1) dissolving a preservative, xylitol and a buffering agent in water to prepare a solution;
(2) dissolving the GLP-1 analog in the above solution, adjusting the pH to the desired pH;
(3) adding a stabilizer to the above solution to obtain the pharmaceutical formulation comprising the GLP-1 analog at a concentration from 0.1 mg/ml to 25 mg/ml, a buffer at a pH from 7.5 to 9.0, a stabilizer at a concentration from 0.001% to 0.5% (m/v), xylitol at a concentration from 0.5% to 10% (m/v), and a preservative at a concentration from 0.1 mg/ml to 10 mg/ml.
